(19) United States
(12) Patent Application Publication
(10) Pub. No.: US 2022/0333144 A1
(43) Pub. Date: Oct. 20, 2022

(54) RECOMBINANT MICROORGANISMS FOR IN VIVO PRODUCTION OF SULFATED GLYCOSAMINOGLYCANS

(71) Applicants: Abinaya BADRI, Troy, NY (US); Mattheos AG KOFFAS, Niskayuna, NY (US); Robert John LINHARDT, Albany, NY (US); Asher J. WILLIAMS, Boston, MA (US)

(72) Inventors: Abinaya BADRI, Troy, NY (US); Mattheos AG KOFFAS, Niskayuna, NY (US); Robert John LINHARDT, Albany, NY (US); Asher J. WILLIAMS, Boston, MA (US)

(73) Assignee: Rensselaer Polytechnic Institute, Troy, NY (US)

(21) Appl. No.: 17/641,675

(22) PCT Filed: Sep. 10, 2020
(86) PCT No.: PCT/US2020/050056

§ 371 Date: Mar. 9, 2022

Related U.S. Application Data
(60) Provisional application No. 63/076,442, filed on Sep. 10, 2020.
(60) Provisional application No. 62/898,243, filed on Sep. 10, 2019.

(51) Int. Cl. C12P 19/26; C12N 1/20; C12N 15/70; C12N 9/10; C12N 15/113

(52) U.S. Cl. CPC C12P 19/26; C12N 1/205; C12N 15/70; C12N 9/13; C12Y 208/02005; C12Y 208/02017; C12N 15/1137; C12R 2001/19

(57) ABSTRACT

In order to produce chondroitin sulfate in an animal-free manner, engineered E. coli host cells were modified so as to reduce expression of an endogenous gene for fructosyltransferase (kfoE); reduce expression of an endogenous gene for 3′-phosphoadenosine-5′-phosphosulfate reductase (cysH); and express one or more exogenous sulfotransferases. Expression of proteins forming ATP-binding cassette transporters were also reduced to limit export of glycosaminoglycans from the cells. The recombinant microorganisms are able produce all three components identified for chondroitin sulfate production—chondroitin, sulfate donor, and sulfotransferase. These modified E. coli are capable of complete, essentially one-step biosynthesis of chondroitin sulfate at a variety of sulfation levels from simple microbial media components and glucose. This is a major advantage over current production methods that depend on the natural distribution of chondroitin sulfate types in the animal tissue.

### CROSS REFERENCE TO RELATED APPLICATION(S)

This application is a national stage patent application filing of International Application No. PCT/2020/050056, filed Sep. 10, 2020, which claims the benefit of U.S. Provisional Application Nos. 63/076,442, filed Sep. 10, 2020, and 62/898,243, filed Sep. 10, 2019, which is incorporated by reference as if disclosed herein in its entirety.

### STATEMENT REGARDING FEDERALLY SPONSORED RESEARCH OR DEVELOPMENT

This invention was made with government support under grant no. CBET-1604547 awarded by the National Science Foundation. The government has certain rights in the invention.

### BACKGROUND

Glycosaminoglycans (GAGs) are polysaccharides that include repeating units of hexuronic acid derivatives, e.g., glucuronic acid, iduronic acid, etc., and hexosamine derivatives, e.g., N-acetyl/N-sulfo glucosamine/galactosamine. FIG. 1 shows pathways forming the precursors for different GAG backbones. Post-polymerization modifications, e.g., addition of sulfate groups, epimerization, deacetylation, are made on these compounds. Based on these modifications, each of these GAG families can be further classified. For example, sulfation of chondroitin in the 4th/6th/4th and 6th carbon positions of N-acetyl galactosamine give rise to chondroitin sulfates (type A/C/E respectively). These functional groups determine the specific interactions with proteins and hence contribute to the biological roles of these compounds.

Sulfated GAGs like heparin, heparan sulfates, and chondroitin sulfates constitute an essential and abundant component of the extracellular matrix in higher eukaryotes. These GAGs serve as important pharmaceuticals, e.g., to treat osteoarthritis, to improve liver function, lower blood sugar, inhibit tumor metastasis, etc., and have also been utilized as thickeners, preservatives, and in drug delivery applications. For example, since the 1940s, heparin has predominated as the primary anticoagulant used in medicine.

Chondroitin sulfate is extensively prescribed in human and veterinary joint health. Chondroitin sulfate is composed of [→4)-β-D-GlcA-(1→3)-β-D-GalNAc-(1→] repeating disaccharide units with various combinations of sulfation and epimerization generating different types. Complex chondroitin sulfate structures in proteoglycans have myriad functional group patterns that allow specific interactions with biomolecules. Such interactions regulate many important cellular processes, including differentiation and development, and determine the role of chondroitin sulfate in health and disease. For example, specific patterns of fructosylated chondroitin sulfate from sea cucumbers have been shown to possess anti-obesity, anti-diabetic, and immunomodulatory activities.

Due to their presence in animals, GAGs are currently commercially manufactured by extraction from animal tissues, primarily from bovine trachea and porcine intestinal mucosa, as well as from chicken, fish, sharks, etc. Prime producers of pig and cattle, such as China, dominate the manufacturing and marketing of GAGs. These sulfated polysaccharides occur as mixtures in tissues with individual components varying slightly in stereochemistry, length, and sulfation pattern. Such small analytical differences result in remarkably distinct biological function and in vivo behavior; they also make their adulteration very hard to detect. Contamination incidents like the heparin adulteration crisis of 2008 and the FDA's warning about questionable crude GAG sources in 2017 have evoked a major conversation about the deficiencies of current production methods, regulatory practices, and analytical detection methods of adulterants/contaminants in GAGs.

GAGs also have complicated structures necessitating sophisticated analytical instrumentation for verifying their purity. GAG activity and specificity are dependent upon their functional group pattern. Specific interactions of GAGs with important biomolecules bring about their physiological roles like anticancer and anti-diabetic properties. The potentials of such properties have created additional demands for the sustainable availability of pure, chemically-defined GAGs. Difficulties in downstream purification, complex and expensive quality control steps, risk of cross-viral contaminations, non-sustainability and inhomogeneity in GAGs from animal tissues, and cultural trends against animal-sourced products are all key forces driving innovation in GAG manufacturing towards sustainable, microbial-based processes.

While such methods are unsustainable and prone to contamination, animal-free production methods have yet to emerge as competitive alternatives due to complexities in scale-up, requirement for multiple stages and cost of co-factors and purification. Chemical synthesis methods are not only tedious and involve multiple steps, but are also difficult to scale up. Synthesis from mammalian cell cultures is also not ideal due to the complexity of handling, high cost of media, low cell densities that can be achieved, and interference from other GAG pathways. Complete microbial synthesis of GAGs holds great promise as it represents a simplified, sustainable process for production of structurally homogeneous GAGs. However, this has not been practically accomplished. Systematic studies identifying favorable, physiological activities of specific GAGs are also severely limited by the availability of pure sample.

### SUMMARY

Some embodiments of the present disclosure are directed to a method for producing sulfated glycosaminoglycans including cultivating a modified bacterium in a culture medium, the bacterium is modified so as to reduce expression of an endogenous gene for fructosyltransferase (kfoE); reduce expression of an endogenous gene for 3′-phosphoadenosine-5′-phosphosulfate reductase (cysH); and express one or more exogenous sulfotransferases. In some embodiments, the bacterium is modified so as to delete the endogenous genes for kfoE, cysH, or combinations thereof. In some embodiments, the bacterium is modified so as to reduce expression of proteins forming ATP-binding cassette transporters to reduce glycosaminoglycans export from the bacterium. In some embodiments, the ATP-binding cassette transporter proteins having reduced expression include KpsT, KpsM, KpsD, KpsE, or combinations thereof. In some embodiments, the method includes extracting a product from the culture medium, the product including sulfated glycosaminoglycans evolved from the modified bacterium. In some embodiments, the method includes isolating sulfated glycosaminoglycans from the product. In some embodiments, the modified bacterium is a modified E. coli K4 strain.

In some embodiments, cultivating a modified bacterium in a culture medium includes inducing the modified bacterium at about 0.6 OD₆₀₀ and expressing the modified bacterium at a temperature of about 16° C. In some embodiments, inducing the modified bacterium at about 0.6 OD₆₀₀ includes an inducer concentration above about 0.5 mM. In some embodiments, cultivating a modified bacterium in a culture medium includes inducing the modified bacterium at about 1.0 OD₆₀₀ and expressing the modified bacterium at a temperature of about 20° C.

In some embodiments, the one or more exogenous sulfotransferases include a chondroitin-4-O-sulfotransferase, a chondroitin-6-O-sulfotransferase, a chondroitin-4,6-O-sulfotransferase, or combinations thereof. In some embodiments, the one or more exogenous sulfotransferases include one or more proteins according to SEQ. ID. NO.: 1, SEQ. ID. NO.: 2, SEQ. ID. NO.: 3, or SEQ. ID. NO.: 4.

Some embodiments of the present disclosure are directed to a method of producing chondroitin sulfate including providing an E. coli host cell, cultivating the E. coli host cell under conditions to preferentially produce chondroitin sulfate, and recovering chondroitin sulfate from the E. coli host cell. In some embodiments, the E. coli host cell being modified so as to reduce expression of an endogenous gene for 3 ‘-phosphoadenosine-5’-phosphosulfate reductase (cysH) and express one or more exogenous sulfotransferases. In some embodiments, the E. coli host cell is modified so as to reduce expression of an endogenous gene for fructosyltransferase (kfoE). In some embodiments, the E. coli host cell is modified so as to delete the endogenous genes for kfoE, cysH, or combinations thereof. In some embodiments, the E. coli host cell is a modified E. coli K4 strain. In some embodiments, the E. coli host cell is a modified E. coli MG1655 strain.

In some embodiments, the one or more exogenous sulfotransferases include a chondroitin-4-O-sulfotransferase, a chondroitin-6-O-sulfotransferase, a chondroitin-4,6-O-sulfotransferase, or combinations thereof. In some embodiments, the one or more exogenous sulfotransferases include one or more proteins according to SEQ. ID. NO.: 1, SEQ. ID. NO.: 2, SEQ. ID. NO.: 3, or SEQ. ID. NO.: 4.

In some embodiments, cultivating a modified bacterium in a culture medium includes inducing the modified bacterium at about 0.6 OD₆₀₀ at an inducer concentration above about 0.5 mM and expressing the modified bacterium at a temperature of about 16° C. In some embodiments, cultivating a modified bacterium in a culture medium includes inducing the modified bacterium at about 1.0 OD₆₀₀ and expressing the modified bacterium at a temperature of about 20° C.

Some embodiments of the present disclosure are directed to a modified bacterium for producing chondroitin sulfate including one or more exogenous genes encoding for a chondroitin-4-O-sulfotransferase, a chondroitin-6-O-sulfotransferase, a chondroitin-4,6-O-sulfotransferase, or combinations thereof. In some embodiments, the bacterium has been modified to reduce expression of: an endogenous gene for 3′-phosphoadenosine-5′-phosphosulfate reductase (cysH), an endogenous gene for fructosyltransferase (kfoE), and an endogenous gene encoding one or more proteins that form ATP-binding cassette transporters to reduce glycosaminoglycans export from the bacterium. In some embodiments, the endogenous genes for 3′-phosphoadenosine-5′-phosphosulfate reductase (cysH), fructosyltransferase (kfoE) are deleted in the bacterium. In some embodiments, the modified bacterium is a modified E. coli K4 strain.

### BRIEF DESCRIPTION OF THE DRAWINGS

The drawings show embodiments of the disclosed subject matter for the purpose of illustrating the invention. However, it should be understood that the present application is not limited to the precise arrangements and instrumentalities shown in the drawings, wherein:

FIG. 1 is a diagram of a glycosaminoglycan (GAG) production pathways;

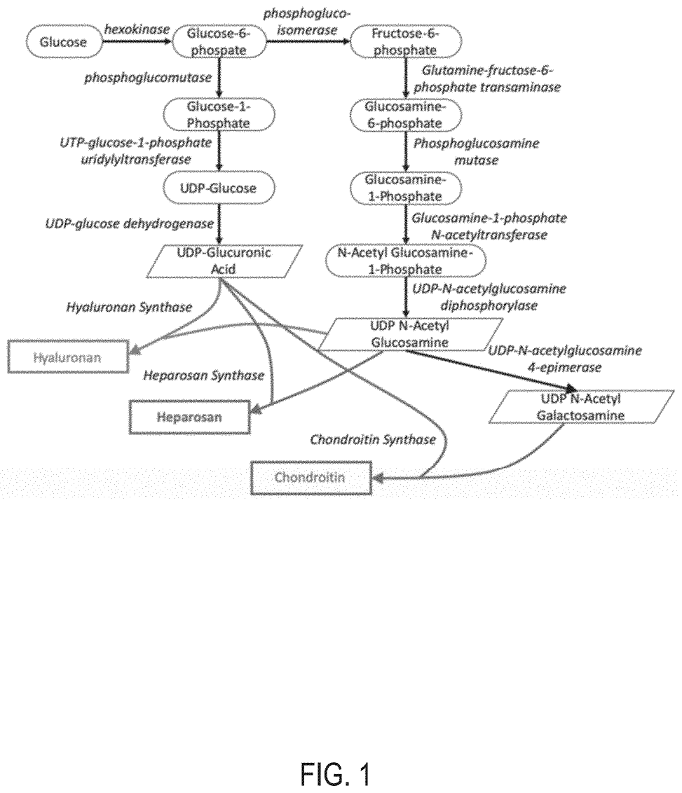

FIG. 2 is a chart of a method for producing sulfated GAGs according to some embodiments of the present disclosure;

FIG. 3 is a schematic representation of cellular transmembrane transport of glycosaminoglycans;

FIG. 4 is a graph of showing the effect of induction, inducer concentration, and expression temperatures on the sulfation of GAGs by modified bacteria according to some embodiments of the present disclosure; and

FIG. 5 is a chart of a method for producing chondroitin sulfate according to some embodiments of the present disclosure.

### DETAILED DESCRIPTION

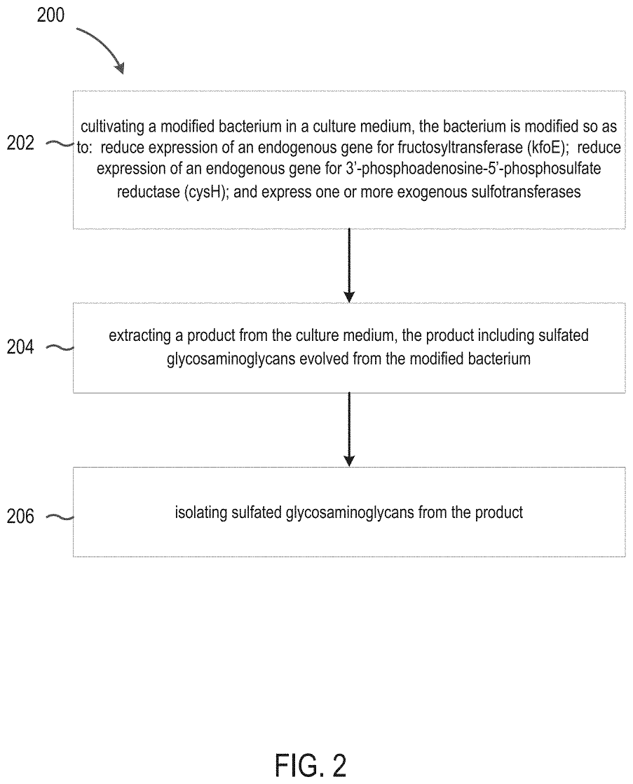

Referring now to FIG. 2, some aspects of the disclosed subject matter include a method 200 for producing sulfated glycosaminoglycans (GAGs). In some embodiments, the sulfated GAG is chondroitin sulfate. In some embodiments, the sulfated GAG is a synthetic polysaccharide that is substantially functionally equivalent to chondroitin sulfate. In some embodiments, the sulfated GAG has greater than about 85%, 90%, 95%, or 99% structural homology with chondroitin sulfate.

At 202, a bacterium is cultivated in a culture medium. In some embodiments, the bacterium is a gram-negative bacteria. In some embodiments, the bacterium is modified to reduce expression of one or more genes. In some embodiments, the bacterium is modified to delete one or more genes. In some embodiments, the bacterium is modified to increase expression of one or more endogenous genes. In some embodiments, the bacterium is modified to express one or more exogenous genes. In some embodiments, the bacterium is a modified E. coli strain. In some embodiments, the bacterium is a modified E. coli K4 strain, modified E. coli MG1655 strain, or combinations thereof.

In some embodiments, the bacterium is modified to reduce expression of an endogenous gene for fructosyltransferase (kfoE). Fructosyltransferase is an enzyme involved in the fructosylation of chondroitin's d-glucuronic acid residues at the 3-position. Without wishing to be bound by theory, this fructosylation adversely interferes with the sulfation of chondroitin to chondroitin sulfate, which is devoid is fructosyl groups in some embodiments of the present disclosure. Thus, by reducing the expression of kfoE, production and sulfation of chondroitin backbone is favored over fructosylated chondroitin polymer in the modified bacterium. In some embodiments, the bacterium is modified to delete kfoE. In some embodiments where the bacterium is a modified E. coli MG1655 strain, the strain does not include an endogenous kfoE gene, and thus deletion or reduced expression may not be necessary.

In some embodiments, the bacterium is modified to favor accumulation of 3′-phosphoadenosine-5′-phosphosulfate (PAPS). In some embodiments, the bacterium is modified to favor intracellular accumulation of PAPS. PAPS is a universal sulfate donor involved in most biological sulfation processes. PAPS biosynthesis is a subset of the ubiquitous cysteine/methionine biosynthetic pathways, and hence, is present in almost all cell types, including E. coli. However, PAPS biosynthesis pathways are not necessarily sufficiently active to provide the PAPS concentrations that can facilitate high yields of sulfated products. In some embodiments, the bacterium is modified to reduce expression of an endogenous gene for 3′-phosphoadenosine-5′-phosphosulfate reductase (cysH). Without wishing to be bound by theory, cysH competes with sulfotransferases to reduce PAPS to inorganic sulfite. Thus, by reducing the expression of cysH, a reserve of PAPS available to be acted upon by sulfotransferases is allowed to accumulate. In some embodiments, the bacterium is modified to reduce expression of cysH.

In some embodiments, the bacterium is modified to increase expression of endogenous sulfotransferases, express one or more exogenous sulfotransferases, or combinations thereof. In an exemplary embodiment, sulfation of chondroitin backbone is catalyzed in the bacterium by chondroitin sulfotransferases. Without wishing to be bound by theory, different chondroitin sulfotransferases give rise to different forms of chondroitin sulfate. As competition with cysH for PAPS is reduced as described above, the expressed sulfotransferases are freed up to act on the accumulated PAPS and facilitate higher yields of chondroitin sulfate. In some embodiments, the sulfotransferases include a chondroitin-4-O-sulfotransferase, a chondroitin-6-O-sulfotransferase, a chondroitin-4,6-O-sulfotransferase, or combinations thereof. In some embodiments, the sulfotransferases are encoded by a nucleic acid sequence according to SEQ. ID. NO.: 1, SEQ. ID. NO.: 2, SEQ. ID. NO.: 3, or SEQ. ID. NO.: 4. In some embodiments, the sulfotransferases are encoded by a nucleic acid sequence having greater than about 85%, 90%, 95%, or 99% sequence homology with SEQ. ID. NO.: 1, SEQ. ID. NO.: 2, SEQ. ID. NO.: 3, SEQ. ID. NO.: 4, or combinations thereof.

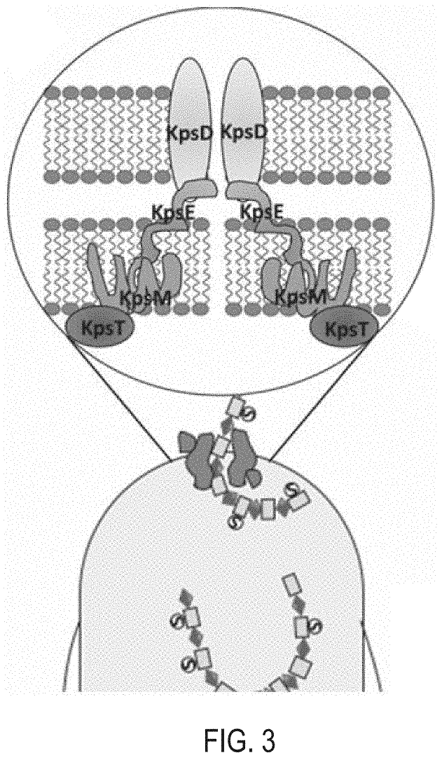

In some embodiments, the bacterium is modified to limit transmembrane transport of GAGs to the extracellular environment surrounding the bacterium. In some embodiments, the bacterium is modified to limit transmembrane transport of unsulfated chondroitin to the extracellular environment surrounding the bacterium. Referring now to FIG. 3, and without wishing to be bound by theory, a currently accepted mechanism of GAG transport in E. coli K4 involves an ATP-binding cassette transporter complex formed by four proteins, KpsT, KpsM, KpsD and KpsE. KpsT is an ATPase that complexes with an inner membrane permease, KpsM. KpsD and KpsE each form dimeric periplasm and membrane spanning complexes that facilitate the export of the polysaccharide. Reduced expression or activity of these complexes impedes export of GAGs, e.g., of the chondroitin backbone, allowing more time for sulfation thereof. In some embodiments, the bacterium is modified to reduce expression of proteins forming ATP-binding cassette transporters. In some embodiments, the ATP-binding cassette transporter proteins having reduced expression include KpsT, KpsM, KpsD, KpsE, or combinations thereof. In some embodiments, one or more genes encoding the ATP-binding cassette transporter proteins are deleted.

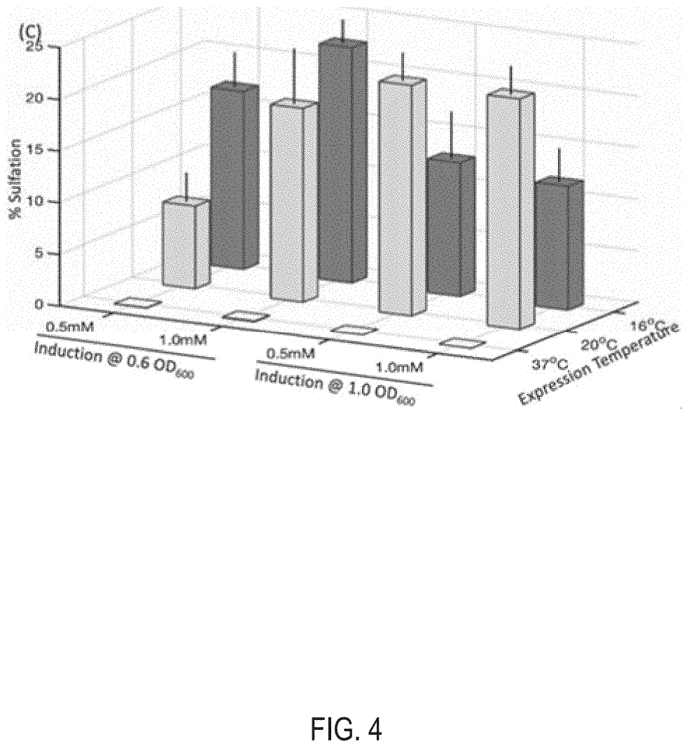

Referring again to FIG. 2, in some embodiments, the culture medium includes a composition suitable to preferentially produce the sulfated GAGs, e.g., chondroitin sulfate. In some embodiments, the modified bacterium is cultivated at about 37° C. In some embodiments, cultivating 202 a modified bacterium in a culture medium includes inducing the modified bacterium at about 0.6 OD₆₀₀ and expressing the modified bacterium at a reduced temperature about of 16° C. In some embodiments, inducing the modified bacterium at about 0.6 OD₆₀₀ includes an inducer concentration between about 0.4 mM and about 1.1 mM. In some embodiments, inducing the modified bacterium at about 0.6 OD₆₀₀ includes an inducer concentration above about 0.5 mM. In some embodiments, inducing the modified bacterium at about 0.6 OD₆₀₀ includes an inducer concentration of about 1 mM. In some embodiments, the inducer is isopropyl-1-thio-β-D-galactopyranoside (IPTG). In some embodiments, cultivating 202 a modified bacterium in a culture medium includes inducing the modified bacterium at about 1.0 OD₆₀₀ and expressing the modified bacterium at a reduced temperature of about 20° C.. In some embodiments, inducing the modified bacterium at about 1.0 OD₆₀₀ includes an inducer concentration between about 0.4 mM and about 1.1 mM. Referring now to FIG. 4, in an exemplary embodiment, maintaining cultivation temperature at 37° C. post-induction resulted in substantially no GAG sulfation irrespective of induction OD₆₀₀ or inducer concentration, indicating potentially poor expression of active sulfotransferase at higher temperatures. Likewise, incubation at lower temperatures from the start of the fermentation resulted in slow growth and also resulted in minimal sulfation. However, dropping the post-induction temperature to express at 16° C. resulted in improved GAG sulfation in cultures induced at 0.6 OD₆₀₀, while expression at 20° C. resulted in improved sulfation for cultures induced at 1.0 OD₆₀₀. In contrast to induction at 1.0 OD₆₀₀, induction at 0.6 OD₆₀₀ made the culture more sensitive to inducer concentration. Overall, this exemplary embodiment demonstrates that fermentation conditions that improved GAG sulfation from ˜19% to ˜23% in modified E. coli K4 strains consistent with the above-identified disclosure

Referring again to FIG. 2, at 204, a product from the culture medium is extracted from the culture medium. In some embodiments, the product includes one or more target compounds. In some embodiments, the one or more target compounds include sulfated glycosaminoglycans evolved from the modified bacterium. In some embodiments, the sulfated GAGs include chondroitin sulfate. At 206, the one or more target compounds are isolated from the product. As used herein, the term “isolated” also includes purifying the target compound to remove unwanted impurities.

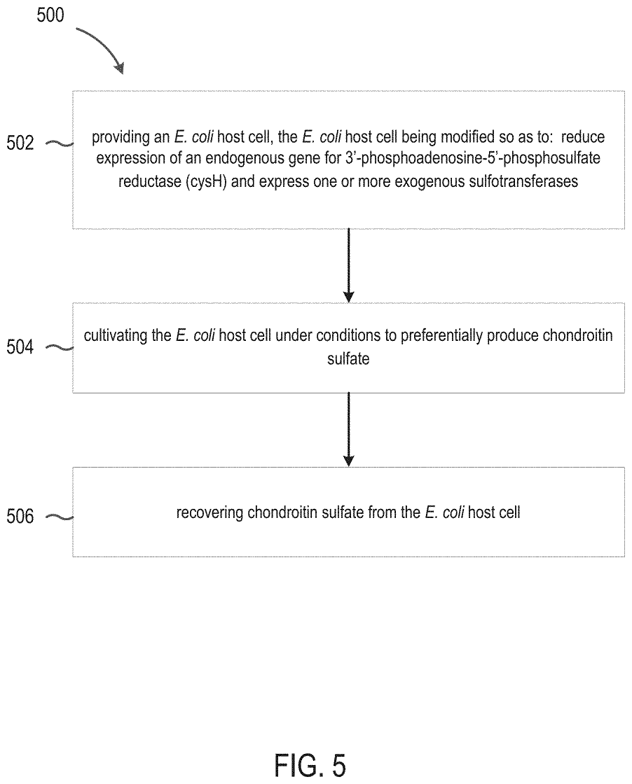

Referring now to FIG. 5, some embodiments of the present disclosure are directed to a method 500 of producing chondroitin sulfate. At 502, an E. coli host cell is provided. In some embodiments, the E. coli host cell has been modified so as to reduce expression of an endogenous gene for 3′-phosphoadenosine-5′-phosphosulfate reductase (cysH). In some embodiments, the E. coli host cell has been modified so as to delete the endogenous genes for cysH. In some embodiments, the E. coli host cell has been modified so as to increase expression of one or more endogenous sulfotransferases. In some embodiments, the E. coli host cell has been modified so as to express one or more exogenous sulfotransferases. In some embodiments, the sulfotransferases include a chondroitin-4-O-sulfotransferase, a chondroitin-6-O-sulfotransferase, a chondroitin-4,6-O-sulfotransferase, or combinations thereof. In some embodiments, the sulfotransferases are encoded by a nucleic acid sequence according to SEQ. ID. NO.: 1, SEQ. ID. NO.: 2, SEQ. ID. NO.: 3, or SEQ. ID. NO.: 4. In some embodiments, the sulfotransferases are encoded by a nucleic acid sequence having greater than about 85%, 90%, 95%, or 99% sequence homology with SEQ. ID. NO.: 1, SEQ. ID. NO.: 2, SEQ. ID. NO.: 3, SEQ. ID. NO.: 4, or combinations thereof. In some embodiments, the E. coli host cell has been modified so as to reduce expression of an endogenous gene for fructosyltransferase (kfoE). In some embodiments, the E. coli host cell has been modified so as to delete the endogenous genes for kfoE.

At 504, the E. coli host cell is cultivated under conditions to preferentially produce chondroitin sulfate. In some embodiments, as discussed above, cultivating a modified bacterium includes inducing the modified bacterium at about 0.6 OD₆₀₀ at an inducer concentration above about 0.5 mM and expressing the modified bacterium at a temperature about 16° C. Also as discussed above, in some embodiments, a modified bacterium in a culture medium includes inducing the modified bacterium at about 1.0 OD₆₀₀ and expressing the modified bacterium at a temperature about 20° C. At 506, chondroitin sulfate is recovered from the E. coli host cell, e.g., as a purified product. In some embodiments, the E. coli host cell is a modified E. coli K4 strain. In some embodiments, the E. coli host cell is a modified E. coli MG1655 strain. In some embodiments, the E. coli host cell is a modified E. coli K4 strain, a modified E. coli MG1655 strain, or combinations thereof.

Some embodiments of the present disclosure are directed to a modified bacterium for producing chondroitin sulfate. In some embodiments, the bacterium is modified to include or increased expression of one or more genes encoding for a chondroitin-4-O-sulfotransferase, a chondroitin-6-O-sulfotransferase, a chondroitin-4,6-O-sulfotransferase, or combinations thereof. In some embodiments, the bacterium is modified to reduce expression of: an endogenous gene for 3′-phosphoadenosine-5′-phosphosulfate reductase (cysH) and endogenous genes encoding one or more proteins that form ATP-binding cassette transporters (to reduce glycosaminoglycans export from the bacterium). In some embodiments, the bacterium is modified to reduce expression of: an endogenous gene for 3′-phosphoadenosine-5′-phosphosulfate reductase (cysH), an endogenous gene for fructosyltransferase (kfoE), and endogenous genes encoding one or more proteins that form ATP-binding cassette. In some embodiments, the bacterium is modified to delete the endogenous genes for cysH. In some embodiments, the bacterium is modified to delete the endogenous genes for kfoE. In some embodiments, the modified bacterium is a modified E. coli strain. In some embodiments, the modified bacterium is a modified E. coli K4 strain. In some embodiments, the modified bacterium is a modified E. coli MG1655 strain. In an exemplary embodiment, chondroitin production in MG1655ΔcysH(DE3) was enabled by expression of the K4 genes, kfoC, kfoA and kfoF through the plasmid pETM6-PCAF. Furthermore, the assembly of all three components in MG1655ΔcysH(DE3) by co-expression of sulfotransferase (pETM6-PCAFSw) led to a high intracellular CS sulfation level of 58%.

### EXAMPLES

Reagents, bacterial strains and plasmids. LB Broth (Lennox), salts and reagents for super optimal broth with catabolite repression (SOC) were procured from MilliporeSigma (St. Louis, Mo.). BD Difco™ M9 minimal media salts and BD Bacto™ casamino acids were procured from BD Biosciences (Franklin Lakes, N.J.). Standard lithium salt of 3′-phosphoadenosine-5′-phosphosulfate (PAPS) and reagents for disaccharide labeling were bought from MilliporeSigma (St. Louis, Mo.). CS disaccharide standards were purchased from Iduron (Manchester, UK). High performance liquid chromatography (HPLC)-grade solvents and salts used to prepare mobile phases were procured from Fisher Scientific (Springfield, N. J.).

Bacterial strains used in this study are E. coli DH5α, E. coli BL21Star(DE3), E. coli K-12 MG1655(DE3) and E. coli K4. ePathBrick vector pETM6 was used to overexpress Chondroitin and PAPS metabolic pathway genes. pETM6 and pET32LIC were used to express chondroitin-4-O-sulfotransferase and its mutants. Transformants were selected using ampicillin resistance that is conferred by the vector backbone, followed by colony polymerase chain reaction (PCR) and Sanger sequencing. CRISPRi repression relied on pdCas9 plasmid carrying a nuclease-null Cas9 from Streptococcus pyogenes and a sgRNA scaffold.

Construction of E. coli K4 ΔkfoE. E. coli K4 (Serovar 05:K4:H4) was engineered for the synthesis chondroitin. The fructosyltransferase encoded by kfoE was deleted by λ red recombineering techniques resulting in K4 ΔkfoE. The FRT-flanked kanamycin resistance cassette was PCR amplified from pKD4 by deletion primers with 40 nucleotides homologous regions near kfoE on the genome. The PCR product was purified by PCR cleanup kit (Cycle Pure Kit, Omega) and transformed into the λ red recombinase expressing E. coli K4. Positive knockout strains were screen by colony PCR and the transformed with pCP20, which expressed the flippase recombination enzyme, to remove the antibiotics resistance marker.

T7 RNA polymerase gene with lacUV5 promoter was integrated into the LacZ position in the E. coli K4 genome. Briefly, a small fragment of “landing-pad” with a tetracycline resistant marker was amplified from pTKS/CS with flanking 40 bp homologous regions of LacZ. Transformation of this purified linear DNA into K4 ΔkfoE expressing λ red recombinase enabled recombination and integration. Positive colonies were verified for successful integration of the landing-pad. Next, T7 RNA-polymerase gene was cloned into the pTKIP vector and transformed into K4 ΔkfoE strains with landing-pad integration harboring pKDRED expressing yeast restriction enzyme I-SecI. Induction of I-SecI cuts at the landing pad and also cleaves out the T7-RNA-polymerase insert from pTKIP which is integrated into the landing pad region with the aid of λ red recombinases. This resulted in strain E. coli K4 ΔkfoE (DE3).

Deletion of PAPS reductase from K4 and MG1655. The cysH gene in E. coli encoding for PAPS reductase, was deleted using λ red recombinase. Briefly, a linear kanamycin resistance cassette with 40-bp homology arms to the two ends flanking the chromosomal cysH gene was amplified from pKD4 and transformed into host expressing recombinases from pKD46. On recombination, correctly deleted colonies were selected based on: kanamycin resistance; loss of ability to grown on M9 media (without casamino acids); size of chromosomal amplicon around the cysH gene region; and Sanger sequencing of the amplicon. Using this method, the cysH gene was deleted from E. coli strains K4ΔkfoE(DE3) and MG1655(DE3).

Growth. Plate cultures of E. coli were grown by streaking glycerol stocks (frozen) onto LB agar plates with appropriate antibiotics. Starter cultures (5 mL) were grown in LB broth by shaking with antibiotics at 37° C. in 14 mL culture tubes until growth reached OD₆₀₀ of 0.6-0.8 (about 6 hours). Flask cultures of chondroitinase and sulfotransferase producing strains were grown in 1 L of M9 medium supplemented with 80 μg/mL ampicillin in PYREX Fernbach Culture Flasks (Corning Life Sciences). Flask cultures of chondroitin sulfate producing strains were grown in 125 mL Erlenmeyer flasks by inoculating 1% starter culture in 25 mL of M9 media supplemented with 1% glucose, 1% casamino acids and including the appropriate antibiotics. Cellular growth was estimated using optical density of culture at 600 nm in a Biotek plate reader. Cells were grown at 37° C. until reaching an OD₆₀₀ of 0.6 and induced with 1 mM isopropyl-1-thio-β-D-galactopyranoside (IPTG), after which growth was continued at either 16° C. for 24 h or 20° C. for 12 h or 37° C. for 10 h. All liquid cultures were incubated in a rotary air shaker (NewBrunswick Scientific Innova 44R) at 37° C., 225 rpm. All CS-producing flask experiments were performed in triplicate.

Repression of PAPS reductase and cellular GAG export using CRISPRi. CRISPRi was used to repress the expression of three genes—cysH encoding PAPS reductase, kpsM encoding the permease component of the capsular export complex and kpsT encoding the ATPase component for the capsular transport protein. pdCas9-mCherry was cloned to incorporate spacer sequences into BsaI sites (golden gate cloning). Spacer sequences were selected based on the region just before the start codon of the genes with the 5′-NGG PAM sequence for (d)Cas9. Successful clones were selected based on chloramphenicol resistance, colony color and sanger sequencing.

Computational Protein Redesign of Sulfotransferase. The PROS S protein engineering server was used to identify mutations to improve the sulfotransferase. PROS S predicts mutations that improve protein stability through modification of protein features such as core packing, surface polarity, and backbone rigidity. A human chondroitin-4-O-sulfotransferase sequence with a 60 amino acid truncation in the N-terminus. A homology model structure was built in the Molecular Operating Environment (MOE) software suite (Chemical Computing Group ULC, (Montreal, QC, Canada)) using the structure of the sulfotransferase domain from Synechococcus PCC 7002 Olefin Synthase (PDB code: 4GOX) as a template. Sequence alignment was generated between reference chondroitin sulfate and 4GOX to assess the similarity between the two sequences. Homology modeling tool in MOE generated 10 models with the following parameters enabled: C-terminal and N-terminal outgap modeling, automatic disulfide bond detection and side-chain sampling set at 300K using an Amber 10:EHT force field. Structural alignments and the Ramachandran statistics calculated for the models were used to assess how well the predicted structure conformed to the previously published 4GOX structure and generally well-folded proteins.

Sulfotransferase mutant expression and purification. The three PROSS-predicted mutants of chondroitin sulfate, designated as S_{M1}, S_{M2}, and S_{M4}, were examined for improved activity in E. coli. Mutants S_{M1} and S_{M2} were derived from chondroitin sulfate (S_{W}) in pET32LIC through multiple rounds of site-directed mutagenesis, while the SM4 gene was synthesized by IDT. The genes were cloned into the BamHI and Xhol sites of a pET32LIC vector with N-terminal thioredoxin (Trx) tag (to increase protein solubility) and His-6x tag (for purification). The fusion proteins were estimated to be ˜53 kDa with and PI value of 6.85 (ExPASy). The constructed plasmids were sequence verified and transformed into E. coli BL21Star (DE3). Overnight culture (20 mL) was centrifuged at 6,800×g for 10 min at 25° C. and the pellet re-suspended in 1 L of M9. Sulfotransferase expression was induced at an OD₆₀₀ of ˜0.8 with 0.2 mM IPTG and the culture was incubated for 16-20 h at 22° C.

Cells were harvested by centrifugation at 5,000×g for 10 min at 4° C. and the pellet were sonicated upon re-suspension in 20 mL of 50 mM Tris-HCl buffer (pH 8.0, 500 mM NaCl, 30 mM imidazole). Cell debris was removed by centrifugation at 16,000×g for 1 h at 4° C. Cell lysate was filtered and applied to a column with Ni-NTA resin (Qiagen) and washed with buffer A (50 mM Tris-HCl 500 mM NaCl, 30 mM imidazole pH 7.5) and eluted with buffer B (50 mM Tris-HCl 500 mM NaCl, 300 mM imidazole pH 7.5). The imidazole was removed by buffer exchange and replaced with storage buffer (50 mM Tris-HCl 500 mM NaCl, 10% glycerol pH 7.5) and kept at −80° C. until needed. S_{W}, SM1, SM2 and SM4 were expressed and purified under identical conditions. The expression level and the purity of the target proteins were verified by SDS-PAGE using a NuPage 10% Bis-Tris Midi gel (Invitrogen).

Analytical Estimations: PAPS using HPLC/UV. On harvesting, cells were pelleted at 4° C. Metabolites, including PAPS, were extracted from the pellet with two 30 min washes of 80% methanol solution at −80° C. Pooled extracts could be stored at −20° C. until further analysis. PAPS concentration in the extract was estimated by HPLC using a 150×2 mm Develosil C-30 RPAqueous column (manufactured by Nomura Chemicals, Japan and purchased from Phenomenex, Inc., USA) in an Agilent LC1260 instrument. Potassium phosphate buffer (100 mM, pH 5.8) and 75% acetonitrile (in H₂O) were used as mobile phases A and B respectively. The samples were run on a 40 min protocol (adapted from Furuno and co-workers) at an overall flow rate of 0.2 mL/min. The gradient program was set as follows: 0% B from 0-10 min; 0-50% B (linear ramp) from 10-12 min; 50% B from 12-17 min; 50-0% B (linear ramp) from 17-20 min and 0% B from 20-40 min. Standard PAPS (detected using PDA detector at 260 nm) diluted in mobile phase A elutes at 3.1 min.

Analytical Estimations: GAG extraction and Disaccharide analysis using LC/MS. Extracellular GAGs produced in each flask culture were recovered in the solution phase (spent media) after centrifugation. Intracellular GAGs were recovered by re-suspending the cell pellet, autoclaving to prepare cell lysate, and centrifuging to recover the soluble phase. Both solutions including extracellular and intracellular GAGs were precipitated with 4 volumes ethanol and stored at −20° C. for 12 h in an explosion-proof freezer. The precipitates were collected, dried, and re-dissolved in 0.2 volume sterile water to generate GAG extracts that were stored at −20° C. until further use.

Extracted GAG solutions (100 μL) were passed through a 3 kDa spin column to remove small molecules and to exchange with digestion buffer (50 mM ammonium acetate, 2 mM CaCl₂) (pH 7.4)). GAG solutions were added to 200 μL of digestion buffer and 20 mU purified chondroitinase ABC (25 mM Tris, 500 mM NaCl, 300 mM imidazole buffer (pH 7.4)) and incubated at 37° C. for 12 h for depolymerization. The resulting disaccharides were passed through a 3-kDa spin-column, then the filtrate was collected and lyophilized. The freeze-dried disaccharide samples were fluorescently labeled by dissolving in 10 μL of a 0.1 M 2-aminoacridone (AMAC) (17:3 of dimethyl sulfoxide:acetic acid (v:v)). After incubation for 10 min at room temperature, the reaction mixture was supplemented with 10 μL of 1 M NaBH₃CN, vortex-mixed, and incubated at 45° C. for 1 h. Samples were centrifuged and the supernatant including the labeled disaccharides was analyzed. The AMAC-labeled disaccharides were separated by HPLC on an Agilent Poroshell 120, EC-C18 column (Agilent Technologies, Inc. Wilmington, Del.) using an Agilent 1200 HPLC system with detection by a TSQ Quantum triple quadrupole electron-spray ionization mass spectrometer (Thermo Finnigan, San Jose, Calif.)53. Data were processed to identify disaccharide levels using the Thermo Xcalibur software.

Analytical Estimations: In vitro sulfotransferase assays. Colorimetric activity assay followed a previously published method with some adaptations. The total assay volume was 200 μL, including 100 μL 50 mM 2-(N-morpholino)ethanesulfonic acid (MES) buffer, 20 μL p-nitrophenyl sulfate (PNPS) (20 mM), 20 μL chondroitin (1 mg/mL), 20 μL of 1 mg/mL AST-IV, 20 μL purified C4ST (˜1 mg/mL), and 20 μL PAPS (2.5 mM). The assay solution was mixed, with PAPS added immediately before absorbance measurements were started. The temperature controlled SpectraMax plate reader (Molecular Devise, Sunnyvale, Calif.) was pre-incubated at 37° C., then the formation of PNP was detected at absorbance 400 nm. The reactions were allowed to continue at 37° C. overnight, then processed for disaccharide analysis.

Methods and systems of the present disclosure are advantageous as single microbial cell factories capable of complete, essentially one-step biosynthesis of chondroitin sulfate at a variety of sulfation levels. Wildtype E. coli does not have the natural ability to produce GAGs. However, chondroitin sulfates can be made entirely animal-free via the engineered E. coli strains of the present disclosure, producing chondroitin sulfates from simple microbial media components and glucose. This is a major advantage over current production methods that depend on the natural distribution of chondroitin sulfate types in the animal tissue.

The recombinant microorganisms are able produce all three components identified for chondroitin sulfate production—chondroitin, sulfate donor and sulfotransferase. In this way, intracellular chondroitin sulfate production of ˜14 μg/g dry-cell-weight was achieved with about 55% of the disaccharides sulfated. Apart from enabling more pharmaceutical and cell-culture applications, the present disclosure also decreases purification steps and alleviates viral contamination issues.

Although the disclosed subject matter has been described and illustrated with respect to embodiments thereof, it should be understood by those skilled in the art that features of the disclosed embodiments can be combined, rearranged, etc., to produce additional embodiments within the scope of the invention, and that various other changes, omissions, and additions may be made therein and thereto, without parting from the spirit and scope of the present invention.

What is claimed is:

1. A method for producing sulfated glycosaminoglycans, comprising:
cultivating a modified bacterium in a culture medium, the bacterium is modified so as to:
reduce expression of an endogenous gene for fructosyltransferase (kfoE);
reduce expression of an endogenous gene for 3′-phosphoadenosine-5′-phosphosulfate reductase (cysH); and
express one or more exogenous sulfotransferases;
extracting a product from the culture medium, the product including sulfated glycosaminoglycans evolved from the modified bacterium; and
isolating sulfated glycosaminoglycans from the product.

2. The method according to claim 1, wherein cultivating a modified bacterium in a culture medium includes:
inducing the modified bacterium at about 0.6 OD₆₀₀; and
expressing the modified bacterium at a temperature of about 16° C.

3. The method according to claim 2, wherein inducing the modified bacterium at about 0.6 OD₆₀₀ includes an inducer concentration above about 0.5 mM.

4. The method according to claim 1, wherein cultivating a modified bacterium in a culture medium includes:
inducing the modified bacterium at about 1.0 OD₆₀₀; and
expressing the modified bacterium at a temperature of about 20° C.

5. The method according to claim 1, wherein the one or more exogenous sulfotransferases include a chondroitin-4-O-sulfotransferase, a chondroitin-6-O-sulfotransferase, a chondroitin-4,6-O-sulfotransferase, or combinations thereof.

6. The method according to claim 5, wherein the one or more exogenous sulfotransferases include one or more proteins according to SEQ. ID. NO.: 1, SEQ. ID. NO.: 2, SEQ. ID. NO.: 3, or SEQ. ID. NO.: 4.

7. The method according to claim 1, wherein the bacterium is modified so as to delete the endogenous genes for kfoE, cysH, or combinations thereof.

8. The method according to claim 1, wherein the modified bacterium is a modified E. coli K4 strain.

9. The method according to claim 1, wherein the bacterium is modified so as to:
reduce expression of proteins forming ATP-binding cassette transporters to reduce glycosaminoglycans export from the bacterium.

10. The method according to claim 9, wherein the ATP-binding cassette transporter proteins having reduced expression include KpsT, KpsM, KpsD, KpsE, or combinations thereof.

11. A method of producing chondroitin sulfate, comprising:
providing an E. coli host cell, the E. coli host cell being modified so as to:
reduce expression of an endogenous gene for 3′-phosphoadenosine-5′-phosphosulfate reductase (cysH); and
express one or more exogenous sulfotransferases;
cultivating the E. coli host cell under conditions to preferentially produce chondroitin sulfate; and
recovering chondroitin sulfate from the E. coli host cell.

12. The method according to claim 11, wherein the E. coli host cell is a modified E. coli MG1655 strain.

13. The method according to claim 11, wherein the E. coli host cell is modified so as to reduce expression of an endogenous gene for fructosyltransferase (kfoE).

14. The method according to claim 13, wherein the E. coli host cell is modified so as to delete the endogenous genes for kfoE, cysH, or combinations thereof.

15. The method according to claim 14, wherein the E. coli host cell is a modified E. coli K4 strain.

16. The method according to claim 11, wherein the one or more exogenous sulfotransferases include a chondroitin-4-O-sulfotransferase, a chondroitin-6-O-sulfotransferase, a chondroitin-4,6-O-sulfotransferase, or combinations thereof.

17. The method according to claim 16, wherein the one or more exogenous sulfotransferases include one or more proteins according to SEQ. ID. NO.: 1, SEQ. ID. NO.: 2, SEQ. ID. NO.: 3, or SEQ. ID. NO.: 4.

18. The method according to claim 11, wherein cultivating a modified bacterium in a culture medium includes:
inducing the modified bacterium at about 0.6 OD₆₀₀ at an inducer concentration above about 0.5 mM; and
expressing the modified bacterium at a temperature of about 16° C.

19. The method according to claim 11, wherein cultivating a modified bacterium in a culture medium includes:
inducing the modified bacterium at about 1.0 OD₆₀₀; and
expressing the modified bacterium at a temperature of about 20° C.

20. A modified bacterium for producing chondroitin sulfate, comprising:
one or more exogenous genes encoding for a chondroitin-4-O-sulfotransferase, a chondroitin-6-O-sulfotransferase, a chondroitin-4,6-O-sulfotransferase, or combinations thereof;
wherein the bacterium has been modified to reduce expression of: an endogenous gene for 3′-phosphoadenosine-5′-phosphosulfate reductase (cysH), an endogenous gene for fructosyltransferase (kfoE), and an endogenous gene encoding one or more proteins that form ATP-binding cassette transporters to reduce glycosaminoglycans export from the bacterium.

21. The modified bacterium according to claim 20, wherein the endogenous genes for 3′-phosphoadenosine-5′-phosphosulfate reductase (cysH) and fructosyltransferase (kfoE) are deleted in the bacterium.

22. The modified bacterium according to claim 20, wherein the modified bacterium is a modified E. coli K4 strain.


SEQUENCE LISTING (4 sequences)

SEQ ID NO: 1 - DNA, 879 bp, Homo sapiens
caggaactctacaacccaatccagctggagctctcaaacactgctgtcctgcaccagatgcggcgggaccaggtgacagacacgtgccgagccaacagcgccacaagccgtaagcggagggtgctgacccccaacgacctgaagcacttggtggtggatgaggaccacgagctcatctactgctacgtgcccaaggtggcctgcaccaactggaagcggctcatgatggtcctgaccgggcgggggaagtacagcgaccccatggagatcccggccaacgaggcacacgtctccgccaacctgaagaccctgaaccagtacagcatcccagaaatcaaccaccgcttgaaaagctacatgaagttcctgtttgtccgggagcccttcgagaggctagtgtccgcctaccgcaacaagttcacccagaagtacaacatctccttccacaagcggtacggcaccaagatcatcaaacgccagcggaagaacgccacccaggaggccctgcgcaaaggggacgatgtcaaattcgaggagtttgtggcctatctcatcgacccacacacccagcgggaggagcctttcaacgaacactggcaaaccgtctactcactctgccatccctgccacatccactatgacctcgtgggcaagtacgagacactggaagaggattctaattacgtcctgcagctggcaggagtgggcagctacctgaagttccccacctatgcaaagtctacgagaactactgatgaaatgaccacagaattcttccagaacatcagctcagagcaccaaacgcagctgtacgaagtctacaaactcgattttttaatgttcaattactcagtgccaagctacctgaaattggaataa

SEQ ID NO: 2 - DNA, 876 bp, Artificial Sequence
  Synthetic Polynucleotide
caggaactgtataacccgattcagctggaactgagcaacaccgcggtgctgcatcagatgcgccgcgatcaggtgaccgatacctgccgcgcgaacagcgcgaccagccgcaaacgccgcgtgctgaccccgaacgatctgaaacatctggtggtggatgaagatcatgaactgatttattgctatgtgccgaaagtggcgtgcaccaactggaaacgcctgatgatggtgctgaccggccgcggcaaatatagcgatccgatggaaattccggcgaacgaagcgcatgtgagcgcgaacctgaaaaccctgaaccagtatagcattccggaaattaaccatcgcctgaaaagctatatgaaatttctgtttgtgcgccatccgtttgaacgcctggtgagcgcgtatcgcaacaaatttacccagaaatataacattagctttcataaacgctatggcaccaaaattattaaacgccagcgcaaaaacgcgacccaggaagcgctgcgcaaaggcgatgatgtgaaatttgaagaatttgtggcgtatctgattgatccgcatacccagcgcgaagaaccgtttaacgaacattggcagaccgtgtatagcctgtgccatccgtgccatattcattatgatctggtgggcaaatatgaaaccctggaagaagatagcaactatgtgctgcagctggcgggcgtgggcagcagcctgaaatttccgacctatgcgaaaagcacccgcaccaccgatgaaatgaccaccgaattttttcagaacattagcagcgaacatcagacccagctgtatgaagtgtataaactggattttctgatgtttaactatagcgtgccgagctatctgaaactggaa

SEQ ID NO: 3 - DNA, 876 bp, Artificial Sequence
  Synthetic Polynucleotide
caggaactgtataacccgattcagctggaactgagcaacaccgcggtgctgcatcagatgcgccgcgatcaggtgaccgatacctgccgcgcgaacagcgcgaccagccgcaaacgccgcgtgctgaccccgaacgatctgaaacatctggtggtggatgaagatcatgaactgatttattgctatgtgccgaaagtggcgtgcaccaactggaaacgcctgatgatggtgctgaccggccgcggcaaatatagcgatccgatggaaattccggcgaacgaagcgcatgtgagcgcgaacctgaaaaccctgaaccagtatagcattccggaaattaaccatcgcctgcgcagctatatgaaatttctgtttgtgcgccatccgtttgaacgcctggtgagcgcgtatcgcaacaaatttacccagaaatataacattagctttcataaacgctatggcaccaaaattattaaacgccagcgcaaaaacgcgacccaggaagcgctgcgcaaaggcgatgatgtgaaatttgaagaatttgtggcgtatctgattgatccgcatacccagcgcgaagaaccgtttaacgaacattggcagaccgtgtatagcctgtgccatccgtgccatattcattatgatctggtgggcaaatatgaaaccctggaagaagatagcaactatgtgctgcagctggcgggcgtgggcagcagcctgaaatttccgacctatggcaaaagcacccgcaccaccgatgaaatgaccaccgaattttttcagaacattagcagcgaacatcagacccagctgtatgaagtgtataaactggattttctgatgtttaactatagcgtgccgagctatctgaaactggaa

SEQ ID NO: 4 - DNA, 879 bp, Artificial Sequence
  Synthetic Polynucleotide
caggaactctacaacccatcccagctggagctctcaaacactgctgtcctgcaccagatgcggcgggaccaggtgacagacacgtgccaagccaacagcgccacaagccgtaagcggagggtgctgacccccaacgacctgaagcacttggtggtggatgaggaccacgagctcatctactgctacgtgcccaaggtggcctgcaccaactggaagcggctcatgatggtcctgaccgggcgggggaagtacagcgaccccatggagatcccggccaacgaggcacacgtctccgccaacctgaagaccctgaacgagtacagcatcccagaaatcaaccaccgcttgcgaaactacatgaagttcctgtttgtccggcatcccttcgagaggctagtgtccgcctaccgcaacaagttcacccagaagtacaacacctccttccacaagcggtacggcaccaagatcatcaaacgccagcggaagaacgccacccaggaggccctgcgcaaaggggacgatgtcaaattcgaggagtttgtggcctatctcatcgacccacacacccagcgggaggagcctttcaacgaacactggcaaaccgtctactcactctgccatccctgccacatccactatgacctcgtgggcaagtacgagacactggaagaggatgctaattacgtcctgcagctggcaggagtgggcgactccctgaagttccccacctatggaaagtctacgagaactactgatgaaatgaccacagaattcttccagaacatcagctcagagcaccaaacgcagctgtaccaagtctacaaactcgattttttaatgttcaattactcagtgccaagctacctgaaattggaataa